(19) United States
(12) Patent Application Publication
(10) Pub. No.: US 2003/0226217 A1
(43) Pub. Date: Dec. 11, 2003

(54) Compositions for treating hair comprising a phosphate ester compound and an oxidizing agent

(75) Inventors: Mark Richard Bowes, Surrey (GB); Andrew Allan Burton, Middlesex (GB)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/448,967

(22) Filed: May 30, 2003

(30) Foreign Application Priority Data
May 31, 2002 (GB) 0212516.9

(51) Int. Cl. A61K 7/13

(52) U.S. Cl. 8/405; 8/406; 8/408

(57) ABSTRACT

Compositions suitable for dyeing and bleaching hair comprising a phosphate ester compound and an oxidizing agent. The phosphate ester compound preferably comprises a mixture of monoester phosphates of alkoxylated fatty alcohols containing from about 12 to about 22 carbon atoms with diester phosphates of non-alkoxylated fatty alcohols containing from about 12 to about 22 carbon atoms. Preferably the compositions are in the form of oil-in-water emulsions. The compositions have good rheologies and improve the bleaching and dyeing of hair.

### FIELD

The present invention relates to compositions suitable for bleaching or dyeing hair and methods for bleaching hair or dyeing hair.

### BACKGROUND

Bleaching and dyeing (or coloring) of hair has become increasingly popular over the past years. Younger people may want to change the natural color of their hair to a more fashionable one, while older people may also use dyeing compositions to conceal gray hair. As people grow older, the production of melanin slows, giving more and more gray hair over time.

Melanin can also be purposely altered by chemical treatments to give lighter shades. The lightening is achieved by oxidizing the melanin pigments, usually with an oxidizing agent in alkaline solution, also called bleach. Examples of oxidizing agents that can be used are hydrogen peroxide, potassium, sodium or ammonium salts of perborate or percarbonate, persulfate and percarbamide.

Bleaches are also used during oxidative dyeing treatments. Oxidative (or “permanent”) dye compositions comprise “precursor dyes” which are small molecules capable of diffusing into the hair. These molecules mainly belong to three classes of aromatic compounds: diamines, aminophenols and phenols. They are sufficiently small to diffuse in the hair shaft where, once activated by an oxidizing agent such as hydrogen peroxide, they further react with other precursors to form larger colored complexes. Oxidative hair dye compositions commonly contain, in addition to the dye precursors and a source of peroxide, a variety of additional cosmetic and peroxide stabilizing agents.

Oxidizing agents can activate oxidative dye precursors across a range of pH. However, it is known that enhanced dye oxidation can be achieved via the use of a hair-swelling agent (HSA) that can adjust the pH of the oxidizing solution. Such HSA's further enhance the oxidizing and dyeing process by swelling the hair fibers to aid both the diffusion of the peroxide and dyeing agents into the hair and enabling faster, more thorough dye oxidization and hair dyeing. Preferred hair-swelling agents for adjusting the pH of peroxide hair oxidizing compositions are aqueous alkaline solutions containing ammonia (ammonium hydroxide) or monoethanolamine (MEA).

Oxidative treatments of hair such as bleaching (decoloration, lightening) and oxidative dyeing give good results and are very commonly used. They are however not without drawbacks. For example, the oxidizing agents used for bleaching and oxidative dyeing damage hair to some extent. Persistent efforts are made in the industry to improve the currently existing formulations. New hair dyes compositions are constantly synthesized and tested with the objective of providing compositions with improved rheology, improved colour uptake, gray coverage and colour evenness

It has now been found that specific phosphate ester compounds can be advantageously used in combination with oxidizing agent in bleaching and oxidative hair dye compositions at a pH of from about 8 to about 12. For example, it was found that they could replace usual thickening agents such as acrylate copolymers for providing a suitable rheology to a bleaching composition, with the added benefit of providing better lightening to the bleached hair for the same amount of oxidizing agent. They can also improve the colour uptake, gray coverage and colour evenness of dyeing compositions. They can also improve the efficiency of the dyeing process so that less dye can be used for the same benefit, thereby reducing the cost of the formulation. Finally they can give a suitable rheology to the compositions thereby avoiding dripping and providing a good suspension of aesthetic pigments.

U.S. Pat. No. 6,117,915, Pereira discloses an oil-in-water emulsifier composition comprising a specific blend of mono- and diester phosphates of alkoxylated fatty alcohols and a blend of mono- and diester phosphates on non-alkoxylated fatty alcohols. This emulsifier is said to increases the stability of emulsion while increasing the release of the oil component.

A supplier brochure by Croda Inc, 7 Century Drive Parsippany, N.J., number DS-151 and dated Sep. 19, 2000 relates to the properties of a phosphate compound commercialized under the trademark Crodafos CES.

### SUMMARY

The present invention relates to compositions suitable for bleaching or dyeing hair comprising:

a) a phosphate ester compound selected from:

i) monoester phosphates of alkoxylated fatty alcohols containing from about 12 to about 22 carbon atoms and alkoxylated with from about 1 to about 50 moles of an alkylene oxide (per mole of alkoxylated fatty alcohol), said alkylene oxide being selected from ethylene oxide, propylene oxide and mixtures thereof,

ii) diester phosphates of non-alkoxylated fatty alcohols containing from about 12 to about 22 carbon atoms, and

iii) mixtures thereof,

b) an oxidizing agent suitable for treating hair, preferably hydrogen peroxide, and

c) optionally an oxidative hair dye precursor, and

wherein the pH of the composition is of from about 8 to about 12.

The present invention also relates to method of treating hair comprising the step of contacting hair with such compositions.

These and other features, aspects, and advantages of the present invention will become evident to those skilled in the art from a reading of the present disclosure.

### DETAILED DESCRIPTION

While the specification concludes with claims which particularly point out and distinctly claim the invention, it is believed the present invention will be better understood from the following description.

All cited references are incorporated herein by reference in their entireties. Citation of any reference is not an admission regarding any determination as to its availability as prior art to the claimed invention.

All percentages are by weight of total composition unless specifically stated otherwise. All ratios are weight ratios unless specifically stated otherwise.

As used herein the term “hair” to be treated may be “living” i.e. on a living body or may be “non-living” i.e. in a wig, hairpiece or other aggregation of non-living keratinous fibers. Mammalian, preferably human hair is preferred. However wool, fur and other keratin containing fibers are suitable substrates for the compositions according to the present invention.

A. Phosphate Ester Compound

The compositions of the present invention comprise a phosphate ester compound selected from:

i) monoester phosphates of alkoxylated fatty alcohols containing from about 12 to about 22 carbon atoms and alkoxylated with from about 1 to about 50 moles of an alkylene oxide (per mole of alkoxylated fatty alcohol), said alkylene oxide being selected from ethylene oxide, propylene oxide and mixtures thereof,

ii) diester phosphates of non-alkoxylated fatty alcohols containing from about 12 to about 22 carbon atoms, and

iii) mixtures thereof.

Preferably, the phosphate ester compound is a mixture of said mono-ester phosphates of alkoxylated fatty alcohols with said di-ester phosphates of non-alkoxylated fatty alcohols. More preferably the ratio of mono-ester phosphates of alkoxylated fatty alcohols to di-ester phosphates of non-alkoxylated fatty alcohols is of from 1:10 to 10:1, more preferably of from 1:9 to 3:1. These compositions have good emulsifying properties.

Preferably the compositions of the present invention will be in the form of an oil-in-water emulsion (“cream”).

The phosphate esters of the alkoxylated and non-alkoxylated fatty alcohols of the present invention can be formed by reacting alkoxylated and non-alkoxylated fatty alcohols, respectively, with phosphorous pentoxide (P₂O₅). This esterification process is well known in the art and detailed description of one method of carrying out the process can be found in U.S. Pat. No. 6,177,915, Pereira et al. As detailed in this patent, the reaction will usually result in a blend of mono- and di-phosphate esters. As is well known in the art, the ratio of mono- to di-phosphate esters formed during the esterification process depends on the operating conditions (such as the temperature) and the initial proportion of the reagents.

It is preferred that, when the phosphate ester compound comprises mono-ester phosphates of alkoxylated fatty alcohols, it also comprises di-ester phosphates of alkoxylated fatty alcohols. The alkoxylated fatty alcohols used can be the same for the mono- and di-esters. The phosphate ester compound then comprises a blend of mono- and di-ester phosphates of alkoxylated fatty alcohols containing from about 12 to about 22 carbon atoms and alkoxylated with from about 1 to about 50 moles of an alkylene oxide (per mole of alkoxylated fatty alcohol), said alkylene oxide being selected from ethylene oxide, propylene oxide and mixtures thereof. Preferably the ratio of mono- to di-ester phosphates of alkoxylated fatty alcohol is from about 10:90 to about 90:10, more preferably the ratio is more than about 1:1.

The alkoxylated fatty alcohols used in the esterification process have the formula:

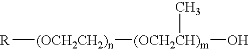

wherein R is selected from branched or unbranched alkyl or alkenyl groups having from about 12 to about 22 carbon atoms, more preferably from about 14 to about 20 carbon atoms, even more preferably from about 16 to about 18 carbon atoms;

n and m are integers independently selected from 0 or a value of from about 1 to about 50, and

at least n or m is different from 0.

Preferably n+m has a value of from about 2 to about 20. The alkoxylated fatty alcohols can be partly ethoxylated and partly propoxylated (n and m≠0) or propoxylated only (n=0) but the preferred alkoxylated fatty alcohols are ethoxylated only (m=0).

It is also preferred that, when the phosphate esters compound comprises di-ester phosphates of non-alkoxylated fatty alcohols, it also comprises mono-ester phosphates of non-alkoxylated fatty alcohols. The alkoxylated fatty alcohols used can be the same for the mono- and di-esters. The phosphate esters compound then comprises a blend of mono- and di-ester phosphates of non-alkoxylated fatty alcohols containing from about 12 to about 22 carbon atoms. Preferably the ratio of mono- to di-ester phosphates of non-alkoxylated fatty alcohol is from about 10:90 to about 90:10, more preferably the ratio is less than about 1:1.

The non-alkoxylated fatty alcohols used for forming the phosphate esters of non-alkoxylated fatty alcohols preferably have the formula R—OH wherein R is selected from branched or unbranched alkyl or alkenyl groups having from about 12 to about 22 carbon atoms, preferably from about 14 to about 20 carbon atoms, even more preferably from about 16 to about 18 carbon atoms.

The compositions of the present invention preferably comprise at least 0.1%, more preferably from about 0.2 to about 20%, even more preferably from 0.3 to 5% by weight of the total composition, of the phosphate esters compound of claim 1.

B. Oxidizing Agent

The compositions of the present invention comprise at least one oxidizing agent suitable for treating hair. Preferred oxidizing agents for use herein are water-soluble peroxygen oxidizing agents. “Water-soluble” as defined herein means that in standard condition at least 0.1 g, preferably 1 g, more preferably 10 g of said oxidizing agent can be dissolved in 1 liter of deionized water in standard conditions. The oxidizing agents are valuable for the initial solubilisation and decolorisation of the melanin (bleaching) and accelerate the polymerization of the oxidative dye precursors (oxidative dyeing) in the hair shaft.

Preferred water-soluble oxidizing agents are inorganic peroxygen materials capable of yielding hydrogen peroxide in an aqueous solution. Water-soluble peroxygen oxidizing agents are well known in the art and include hydrogen peroxide, inorganic alkali metal peroxides such as sodium periodate and sodium peroxide and organic peroxides such as urea peroxide, melamine peroxide, and inorganic perhydrate salt bleaching compounds, such as the alkali metal salts of perborates, percarbonates, perphosphates, persilicates, persulphates and the like. These inorganic perhydrate salts may be incorporated as monohydrates, tetrahydrates, etc. Mixtures of two or more such oxidizing agents can be used if desired. Preferred for use in the compositions according to the present invention is hydrogen peroxide.

In conventional dyeing and bleaching compositions, levels of oxidizing agent are usually of from about 0.1% to about 7% by weight, preferably of from 0.5 to 4% by weight.

C. Oxidative Hair Dye Precursors

The compositions of the present invention may comprise oxidative hair dye precursors. These compounds are well known in the art, and include aromatic diamines, aminophenols and their derivatives (a representative but not exhaustive list of oxidation dye precursor can be found in Sagarin, “Cosmetic Science and Technology”, “Interscience, Special Edn. Vol. 2 pages 308 to 310). Precursors can be used with couplers. Couplers are generally colorless molecules that can form colors in the presence of activated precursors.

The choice of precursors and couplers will be determined by the color, shade and intensity of coloration that is desired. The precursors and couplers can be used herein, singly or in combination, to provide dyes having a variety of shades ranging from ash blonde to black.

Hair dye compositions will generally comprise from about 0.001% to about 10%, preferably from about 0.1% to about 2%, of oxidative dye precursors and couplers.

D. Fatty Alcohols

The compositions of the present invention preferably comprises a fatty alcohol having the formula R′—OH, wherein R′ is selected from branched or unbranched alkyl or alkenyl groups having from about 12 to about 24 carbon atoms. Preferred fatty alcohol is cetearyl alcohol, a mixture of cetyl and stearyl alcohol.

Preferably the fatty alcohols and the phosphonate ester compound (preferably a mixture of dicetyl phosphate and ceteth-10 phosphate) are comprised in the composition in an amount of from about 0.1 to about 20%, preferably of from about 0.5 to about 10%, more preferably of from about 1 to about 5% by weight of the total composition. Such a mixture of cetearyl alcohol, dicetyl phosphate and ceteth-10 phosphate is commercially available from Croda, Inc under the Trademark Crodafos CES.

E. pH Buffering Agents

The compositions of the present invention preferably further comprise a pH buffering agent. The pH buffering agent is present in an amount sufficient to provide the composition with a pH of from about 8 to about 12, preferably of from about 8.5 to about 11.5, more preferably of from about 9 to about 11, even more preferably of from 9.5 to 10.5. Suitable buffering agents are well known in the art and include for example ammonia/ammonium acetate mixture and monoethanolamine (MEA).

F. Additional Components

Moreover, it is also intended that the compositions of the present invention may be complex compositions, which in addition to the ester phosphate compound and oxidizing agent comprise other components that may or may not be active ingredients. This includes, but is not limited to, buffering agents (discussed above), chelants, hair dyeing agents such as oxidative dye precursors (discussed above), non-oxidative dyes, thickeners, solvents, enzymes, anionic, non ionic, amphoteric and cationic surfactants, conditioning agents, carriers, antioxidants, stabilizers, perming actives, perfume, hair swelling agents and/or polymers.

Preferably the viscosity of the composition (Brookfield RVT, Spindle 5, 50 rpm, 25° C.) is at least about 1 Pa.s (1,000 cps), more preferably the viscosity is in the range of from 1 Pa.s to 10 Pa.s.

### EXAMPLES

The following examples further describe and demonstrate the preferred embodiments within the scope of the present invention. The examples are given solely for the purpose of illustration, and are not to be construed as limitations of the present invention since many variations thereof are possible without departing from its spirit and scope.

### Example A

The following example illustrates a bleaching (lightening) composition in the form of a kit comprising a lightening cream (comprising ammonia) and a hydrogen peroxide cream. Both creams are mixed in equal proportion just before use in order to form the bleaching composition that is then immediately applied to the hair to be bleached.

The numerical values given in the next table (“% w/w active on head”) and the following examples relate to the mixture of the two compositions of the kit and express the percentage of active material found in the mixture.

| | |
|---|---|
| Material | % w/w active on head |

Lightening Cream

| | |
|---|---|
| Demineralised Water | 39.6 |
| Ammonium Hydroxide | 2.2 |
| Crodafos CES (RTM) | 3.0 |
| Dipropylene Glycol | 3.0 |
| Acetic Acid | 1.0 |
| EDDS | 0.6 |
| Perfume | 0.3 |
| Ascorbic Acid | 0. |
| Tetrasodium EDTA | 0.1 |
| Sodium Sulphite | 0.1 |

Hydrogen peroxide cream

| | |
|---|---|
| Demineralised Water | 45.2 |
| Hydrogen Peroxide | 1.6 |
| Cetyl Alcohol | 1.13 |
| Stearyl Alcohol | 1.13 |
| Ceteareth 25 | 0.75 |
| Phenoxyethanol | 0.05 |
| Di sodium salt of 1-hydroxyethane-1,1- | 0.05 |
| diphosphonic acid (HEDP) | |
| Pentasodium Pentatate | 0.05 |
| Sodium Benzoate | 0.05 |
| Phosphoric Acid | 0.02 |
| Sodium Stannate | 0.02 |
| Tetrasodium EDTA | 0.02 |

Crodafos CES (RTM) is a mixture of cetearyl alcohol, dicetyl phosphate and ceteth-10 phosphate.

Both creams can be produced by conventional methods well known by the person skilled in the art. These include:

Oil in water process

Phase Inversion process

One-pot process

For instance the water-soluble ingredients can be added to a proportion of the water at the start of the making process at ambient temperature, and allowed to dissolve. The balance of the water is heated to 60-90° C. The fatty components including Crodafos (RTM) and the surfactants are then added to the heated water. A controlled cooling and optional shearing process to form an emulsion pre-mix is then completed. Both pre-mixes are then combined and the remaining ingredients added (e.g. ammonia, hydrogen peroxide, perfume . . . ).

### Example B

The following example illustrates the superiority of phosphate ester compounds in hair dye compositions vs. another class of thickening agent (acrylate copolymers).

A composition similar to the composition of Example A was prepared, with the difference that the phosphate ester compound (Crodafos CES (RTM)) was replaced by 1% Carbopol (RTM) Aqua SF-1 (and water). Carbopol Aqua SF-1 (available from Noveon) is an acrylates copolymer commonly used in hair-care compositions as thickening agent.

Both compositions had similar rheology. Swatches of virgin hair were treated with these compositions in normal dyeing conditions and the resulting color was assessed.

Hair treated with the composition comprising Crodafos CES showed a significantly better lightening than the hair treated with the composition comprising Carbopol (RTM) Aqua SF-1, although the level of oxidizing agent was identical.

This shows that the phosphate ester compounds can provide a hair bleaching composition with a suitable rheology while at the same time increasing the efficiency of the bleaching process.

### Example C

The following example illustrates an oxidative hair dye composition in the form of a kit comprising a dye cream and a hydrogen peroxide cream. The dye cream and the hydrogen peroxide cream are mixed in equal proportion to form the oxidative hair dye composition that is then immediately applied to the hair to be dyed.

| | |
|---|---|
| | % w/w active |
| Material | on head |

Dye cream

| | |
|---|---|
| Demineralised Water | 39.0 |
| Ammonium Hydroxide | 1.5 |
| Crodafos CES | 3.0 |
| Dipropylene Glycol | 3.0 |
| Acetic Acid | 1.0 |
| Trisodium ethyledediaminedissucinic acetate (Na₃EDDS) | 0.6 |
| 4-amino-2-hydroxytoluene | 0.36 |
| Perfume | 0.3 |
| 2-methyl-5-hydroxyethylaminophenol | 0.22 |
| p-Aminophenol | 0.2 |
| Ascorbic Acid | 0.2 |
| Ammonium Thiolactate | 0.15 |
| Tetrasodium EDTA | 0.1 |
| Sodium Sulphite | 0.1 |
| 2,5,Diaminotoluene Sulphate | 0.09 |
| p-Phenylenediamine | 0.06 |
| 2-amino-3-hydroxypyridine | 0.05 |
| 1-phenyl-3-methyl-5-pyrazolone | 0.05 |
| 1-Naphthol | 0.01 |

Hydrogen peroxide cream

| | |
|---|---|
| Demineralised Water | 45.7 |
| Hydrogen Peroxide | 1.05 |
| Cetyl Alcohol | 1.1 |
| Stearyl Alcohol | 1.1 |
| Ceteareth 25 | 0.75 |
| Phenoxyethanol | 0.05 |
| Di sodium salt of 1-hydroxyethane-1,1-diphosphonic acid | 0.05 |
| (HEDP) | |
| Pentasodium Pentatate | 0.05 |
| Sodium Benzoate | 0.045 |
| Phosphoric Acid | 0.02 |
| Sodium Stannate | 0.02 |
| Tetrasodium EDTA | 0.02 |

The dye and hydrogen peroxide cream can be produced by conventional methods well known by the person skilled in the art. These include:

Oil in water process

Phase Inversion process

One-pot process

For instance the water-soluble ingredients can be added to a proportion of the water at the start of the making process at ambient temperature, and allowed to dissolve. The balance of the water is heated to 60-90° C. The fatty components including Crodafos (RTM) and the surfactants are then added to the heated water. A controlled cooling and optional shearing process to form an emulsion pre-mix is then completed. Both pre-mixes are then combined and the remaining ingredients added (e.g. ammonia, hydrogen peroxide, perfume).

In the dye cream, 2% of the dipropylene glycol can be replaced with 1% hexyleneglycol and 1% of ethoxydiglycol in order to speed the dissolution of the dyes during the manufacturing process.

### Example D

The following example illustrates the superiority of phosphate ester compounds in hair lightening compositions vs. another class of thickening agent (acrylate copolymers).

Two compositions similar to the composition of Example C were prepared, with the difference that the phosphate ester compound (Crodafos CES (RTM)) was replaced by 1% Carbopol (RTM) Aqua SF-1 in one case and 2.5% Capigel 98 (RTM) in the other (and water for the rest). Carbopol Aqua SF-1 (available from Noveon) and Capigel 98 (available from SEPPIC) are both acrylates copolymers commonly used in hair dyes compositions as thickening agents.

These compositions and the composition of example C had similar rheology, making them suitable for use as a hair dye. Swatches of virgin hair were treated with these three compositions in normal dyeing conditions and the resulting color was assessed.

Hair treated with the composition comprising Crodafos CES showed a much better color uptake than the hair treated with the other compositions comprising Carbopol (RTM) Aqua SF-1 and Capigel 98 (RTM).

This shows that the phosphate ester compounds can provide a hair dye composition with a suitable rheology while at the same time increasing the efficiency of the dyeing process.

### Example E

The following example illustrates an oxidative hair dye composition in the form of a kit comprising a dye cream and a hydrogen peroxide cream. The creams are mixed just before use.

| | |
|---|---|
| | % w/w active |
| Material | on head |

Dye cream

| | |
|---|---|
| Demineralised Water | 32.4 |
| Crodafos CES | 3.0 |
| Ammonium Hydroxide | 1.5 |
| Dihydroxyethyl soyamine dioleate | 2.1 |
| Stearyl Alcohol | 2.35 |
| Aminofunctional polydimethyl siloxane | 1.5 |
| Cetyl Alcohol | 0.81 |
| Ceteareth 25 | 0.54 |
| Decyl glucoside | 0.5 |
| Steareth 2 | 0.27 |
| di-PEG2 soyamine IPDI | 0.24 |
| Dipropylene Glycol | 3.0 |
| Acetic Acid | 1.0 |
| 4-amino-2-hydroxytoluene | 0.36 |
| p-Aminophenol | 0.20 |
| Ascorbic Acid | 0.20 |
| Ammonium Thiolactate | 0.15 |
| Tetrasodium EDTA | 0.12 |
| Sodium Sulphite | 0.10 |
| Benzyl alcohol | 0.15 |
| Phenoxyethanol | 0.15 |
| p-Phenylenediamine | 0.06 |
| 2-amino-3-hydroxypyridine | 0.05 |

Hydrogen peroxide cream

| | |
|---|---|
| Demineralised Water | 45.7 |
| Hydrogen Peroxide | 1.05 |
| Cetyl Alcohol | 1.13 |
| Stearyl Alcohol | 1.13 |
| Ceteareth 25 | 0.75 |
| Phenoxyethanol | 0.05 |
| Di sodium salt of 1-hydroxyethane-1,1-diphosphonic acid | 0.05 |
| (HEDP) | |
| Pentasodium Pentatate | 0.05 |
| Sodium Benzoate | 0.05 |
| Phosphoric Acid | 0.02 |
| Sodium Stannate | 0.02 |
| Tetrasodium EDTA | 0.016 |

Both cream components can be manufactured as described above.

### Example F

This example illustrates how a phosphate ester compounds such as Crodafos CES (RTM) can increase the color uptake of oxidative hair dye compositions.

A composition similar to the composition of Example E was prepared, with the exception that the amount of Crodafos CES (RTM) was replaced with a identical amount of water. Both compositions were used to dye switches of virgin hair and the resulting colors were measured. It was found that swatches colored with the composition comprising Crodafos (RTM) CES were significantly more intensely red colored than the swatches colored with the composition excluding Crodafos CES.

### Methods of Use

Oxidative dye compositions are usually sold as a kit comprising at least two separate containers: one contains the oxidative dye precursors with the hair-swelling agent (e.g. ammonia) in a suitable carrier (e.g. dye cream or liquid) and the other contains the oxidizing agent in a suitable carrier (e.g. hydrogen peroxide cream or liquid). The consumer prepares the oxidative dye composition immediately before use by mixing both compositions and applies it on hair. After working the mixture a few minutes (to insure uniform application to all of the hair), the oxidative dye composition is allowed to remain on the hair for an amount sufficient for the dyeing to take place (usually about 30 minutes). The consumer then rinses his/her hair thoroughly with tap water and allows it to dry. It is observed that the hair has changed from its original color to the desired color.

Bleaching compositions are usually sold as a kit comprising two or three separate containers. The first contains the hair-swelling agent (e.g. ammonia), the second contains the oxidizing agent and the third (optional) contains a second oxidizing agent (e.g. alkali or ammonium salt of persulphates, percarbonate, perborate). The consumer prepares the bleaching compositions immediately before use by mixing all compositions and applies the mixture on hair (as for the oxidative dye composition) for an amount of time sufficient for the bleaching to take place (usually about 30 mn).

In this kind of kit comprising at least two containers the compositions claimed are representative of the mixture applied on head, and there is no preference on the distribution of the phosphate compound claimed in the different containers.

When present, an optional conditioning agent can be packaged partly or in totality in a third container. In this case, all three compositions can be mixed immediately before use and applied together, or the content of the third container can be applied (after an optional rinse step) as a post-treatment immediately after the oxidative dye or bleaching composition resulting from the mixture of the other containers.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition suitable for bleaching or dyeing hair comprising:
a) a phosphate ester compound selected from:
i) monoester phosphates of alkoxylated fatty alcohols containing from about 12 to about 22 carbon atoms and alkoxylated with from about 1 to about 50 moles of an alkylene oxide (per mole of alkoxylated fatty alcohol), said alkylene oxide being selected from ethylene oxide, propylene oxide and mixtures thereof,
ii) diester phosphates of non-alkoxylated fatty alcohols containing from about 12 to about 22 carbon atoms, and
iii) mixtures thereof,
b) an oxidizing agent suitable for treating hair,
c) and, optionally an oxidative hair dye precursor;
wherein the pH of the composition is of from about 8 to about 12.

2. A composition according to claim 1 wherein the alkoxylated fatty alcohols have the formula: 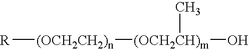
wherein R is selected from branched or unbranched, alkyl or alkenyl groups having from about 12 to about 22 carbon atoms,
n and m are integers independently selected from 0 or a value of from about 1 to about 50, and at least n or m is different from 0.

3. A composition according to claim 1 wherein m=0 and n is a value of from about 2 to about 20.

4. A composition according to claim 1 wherein the non-alkoxylated fatty alcohols have the formula R—OH wherein R is selected from branched or unbranched, alkyl or alkenyl groups having from about 12 to about 22 carbon atoms.

5. A composition according to claim 1 further comprising a fatty alcohol R′—OH wherein R′ is selected from branched or unbranched, alkyl or alkenyl groups having from about 12 to about 24 carbon atoms.

6. A composition according to claim 5 comprising from about 0.1 to about 20%, by weight of the total composition, of a mixture of cetearyl alcohol, dicetyl phosphate and ceteth-10 phosphate.

7. A composition according to claim 1 further comprising diester phosphates of alkoxylated fatty alcohols so that the composition comprises a blend of mono- and di-ester phosphates of alkoxylated fatty alcohols containing from about 12 to about 22 carbon atoms and alkoxylated with from about 1 to about 50 moles of an alkylene oxide (per mole of alkoxylated fatty alcohol), said alkylene oxide being selected from ethylene oxide, propylene oxide and mixtures thereof; and
wherein the ratio of said mono- to di-ester is between about 10:90 and about 90:10.

8. A composition according to claim 1 further comprising monoester phosphates of non-alkoxylated fatty alcohols containing from about 12 to about 22 carbon atoms so that the composition comprises a blend of mono- and di-ester phosphates of non-alkoxylated fatty alcohols containing from about 12 to about 22 carbon atoms and wherein the ratio said of mono- to di-ester is between about 10:90 and about 90:10.

9. A composition according to claim 1 wherein the composition comprises a mixture of said ester phosphates of alkoxylated fatty alcohols with said ester phosphates of non-alkoxylated fatty alcohols.

10. A composition according to claim 9 wherein the ratio of ester phosphates of alkoxylated fatty alcohols to ester phosphates of non-alkoxylated fatty alcohols is of from 1:10 to 10:1.

11. A composition according to claim 1 wherein the viscosity of the composition (Brookfield RVT, Spindle 5, 50 rpm, 25° C.) is at least about 1 Pa.s (1,000 cps).

12. A composition according to claim 1 wherein the pH of the composition is from about 8.5 to about 11.5.

13. A composition according to claim 1 in the form of an oil-in-water emulsion.

14. A method of bleaching or dyeing hair comprising the step of contacting hair with a composition according to claim 1.

15. A kit for dyeing hair comprising at least two compositions packaged in different containers wherein the resulting mixture of said two compositions is a composition according to claim 1.
